(19) United States
(12) Patent Application Publication
(10) Pub. No.: US 2009/0030484 A1
(43) Pub. Date: Jan. 29, 2009

(54) BILATERAL PROSTHESIS SYNCHRONIZATION

(75) Inventor: John Chambers, Mona Vale (AU)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove (AU)

(21) Appl. No.: 12/112,355

(22) Filed: Apr. 30, 2008

(30) Foreign Application Priority Data
Apr. 30, 2007 (AU) 2007902247

(51) Int. Cl. A61F 11/04; A61N 1/36

(52) U.S. Cl. 607/57

(57) ABSTRACT

An arrangement for improving the effectiveness of a bilateral hearing prosthesis system is disclosed. The timing of the prostheses is synchronized allowing the stimulation and other processes to be coordinated so as to minimize interference between the hearing prostheses.

### CROSS-REFERENCE TO RELATED APPLICATIONS

This application claims the benefit of Australian Provisional Application No. 2007902247, entitled “Bilateral Prosthesis Synchronisation,” filed Apr. 30, 2007. This application is hereby incorporated by reference herein.

### BACKGROUND

1. Field of the Invention

The present invention relates generally to hearing prostheses, and more particularly, to synchronization of bilateral prostheses.

2. Related Art

Hearing prostheses are provided to assist individuals who have impaired hearing. Hearing prostheses include cochlear implants, middle ear stimulators, implanted hearing aids, brain stem implants, electro-acoustic devices and other prostheses that provide mechanical, acoustic and/or electrical stimulation to an auditory system of the recipient. More recently, recipients have been provided with two hearing prostheses, one fitted for each auditory system of the recipient. Such combination of hearing prostheses is commonly referred to as a bilateral hearing prosthesis system, or bilateral prostheses.

Bilateral prostheses are generally considered to provide a benefit to the recipient, in that bilateral sound percepts allow in principle for better speech perception by the recipient. It is believed that one important effect is the head shadow effect, essentially allowing the recipient to selectively listen to the side with the better signal-to-noise ratio, generally the side closer to the source of the sound. Inter-aural time delays and level differences may also assist in localizing the sound source, and in separating speech from background noise.

In the case of cochlear implants, the implantable components and associated external components used for bilateral systems are primarily designed to function as independent, monaural systems. It has been observed, however, that the independent operation of such hearing prostheses may be degraded when the hearing prostheses operate in close proximity to each other. Such degradation in operational performance may adversely affect the hearing benefit delivered to the recipient. Such degradation may also affect the quality and integrity of data supplied from the hearing prosthesis, such as telemetry data generated by the hearing prosthesis for clinical and diagnostic use by healthcare professionals.

### SUMMARY

In one embodiment, a method for operating a bilateral hearing prosthesis having first and second hearing prostheses, the first hearing prosthesis configured to perform at least one operation sensitive to interference by emissions generated by the second hearing prosthesis when the second hearing prosthesis performs at least one interfering operation is disclosed. The method comprises synchronizing timing of the sensitive and interfering operations performed by the first and second prostheses; respectively; and performing, based on said synchronized timing, the sensitive and interfering operations such that relative timing of said performance of the sensitive and interfering operations minimizes the interference of the sensitive operations by the emissions generated by the second hearing prosthesis.

In a second embodiment, a hearing prosthesis including a receiver for receiving signals, said receiver communicating with a processor, said processor being operatively adapted to detect synchronizing signals within the received signal, and in response to the synchronizing signals, adjust the timing of operations performed by the prosthesis so as to establish a co-ordinate timing regime with a bilaterally disposed prosthesis is disclosed.

In a third embodiment, a method of communicating between active implanted devices, each said device including a stimulation device adapted to deliver electrical stimuli to the body, and to sense electrical responses to stimuli, wherein the stimulation device is further adapted to send signals which carry information for detection by another implanted device is disclosed.

In a fourth embodiment, a method of communicating between two active implanted devices, wherein such communication is affected by the transmission and reception of vibratory or acoustic signals conveyed through the air and/or body tissues of the recipient is disclosed.

### BRIEF DESCRIPTION OF DRAWINGS

Embodiments of the present invention will now be further described with reference to the accompanying drawings, in which:

FIG. 1 is a perspective view of a bilateral hearing prosthesis system in which embodiments of the present invention may be advantageously implemented;

FIG. 2 is a perspective view of a bilateral hearing prosthesis system in which embodiments of the present invention may be advantageously implemented;

FIG. 3 is a perspective view of a bilateral hearing prosthesis system in which embodiments of the present invention may be advantageously implemented;

FIG. 4 is a perspective view of a bilateral hearing prosthesis system in which embodiments of the present invention may be advantageously implemented;

FIG. 5 illustrates the timing of stimulation and other functions according to one implementation of the present invention;

FIG. 6 illustrates one implementation of synchronization according to the present invention;

FIG. 7 illustrates a second implementation of synchronization; and

FIG. 8 illustrates a third implementation of synchronization;

FIG. 9 illustrates timing issues in a prior art bilateral system, and

FIG. 10 illustrates one implementation of timing for a bilateral system according to an implementation of the present invention.

### DETAILED DESCRIPTION

Aspects of the present invention are generally directed to an improved stimulation timing arrangement in a bilateral hearing prosthesis system, in which the timing of the prostheses is synchronized so that operations (interfering operations) performed by one of the hearing prostheses of the system that may generate emissions that potentially interfere with operations (sensitive operations) of the other hearing prosthesis, and the sensitive operations are performed by the hearing prostheses so as to minimize the potential interference between proximately-located hearing prostheses.

Embodiments of the present invention may be implemented in a bilateral hearing prosthesis system implementing any type of hearing prosthesis now or later developed. Such hearing prostheses may be worn externally by the recipient or may be partially or completely implanted in the recipient. Such hearing prostheses include any combination of the same or similar prostheses such as cochlear implants, middle ear stimulators, implanted hearing aids, brain stem implants, electro-acoustic devices and other prostheses that provide mechanical, acoustic and/or electrical stimulation to an auditory system of the recipient. Embodiments of the present invention are predominantly described with reference to cochlear implants, for example the Freedom™ cochlear implant commercially available from Cochlear Limited, New South Wales, Australia. Such hearing prostheses, as shown for example in FIG. 2, have an implanted receiver/stimulator component 205, including an electrode array 206 and a coil 204 for receiving power and communicating with external component 203. External component 203 comprises a sound processor, one or more microphones (not shown), and a coil 201 for sending power and data to stimulator/receiver 205 via coil 204.

In a broad sense, the present invention controls the relative timing of selected or predetermined or all operations in at least one and both prostheses, so as to minimize inter-prosthesis interference. In one embodiment, prosthesis may perform, execute, initiate (undertake herein) sensitive operations such as communications and signal measurements at a time when interfering operations of the other prosthesis is either suspended or otherwise modified so as to not interfere with such sensitive operations. Alternatively, the undertaking of the sensitive operations is suspended or otherwise modified to avoid interference. In still other embodiments, the relative timing of the sensitive and interfering operations are determined based on performance criteria.

The sensitive operations include, for example, any operations in or by the prosthesis which are susceptible to electrical, magnetic, electromagnetic, mechanical or acoustic interference, and may include for example detecting neural responses, interrogation of clinical and electrode characteristics, and data telemetry transmissions. The interfering operations include, for example, any operations which involve significant transmission, for example transmitting power, data or instructions to an implanted device, and delivering stimuli using an implanted device. The transmissions in principle could be electrical, magnetic, electromagnetic, mechanical or acoustic, depending upon the system concerned.

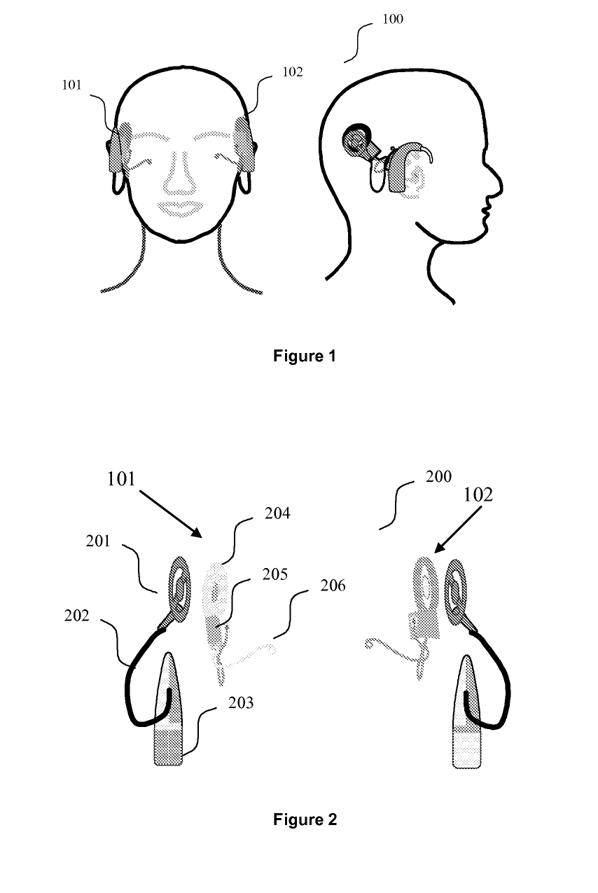

FIG. 1 is a front and side view of a person wearing two hearing prostheses, namely cochlear implants, of a bilateral hearing prosthesis system 100. Bilateral system 100 comprises cochlear implant 101 located on the right side of the recipient's head and cochlear implant 102 located on the left side of the recipient's head. Cochlear implant 101 and 102 may be the same or similar cochlear implants.

FIG. 2 is a perspective view of one embodiment of cochlear implants 101 and 102. Taking cochlear implant 101 as an example, the cochlear implant comprises an external sound processor 203 electrically connected via a cable 202 to an induction coil 201. Stimulation data and electrical power are conveyed electromagnetically from coil 201 to coil 204 associated with implanted stimulator unit 205. Electrical stimuli intended to evoke sound percepts are delivered to the recipient via one or more electrode systems 206. Electrode system 206 comprises an elongate carrier member configured to be inserted into the recipient's right cochlea, and an array of one or more electrodes disposed on the distal end of the carrier member. Cochlear implant 102 is effectively the same as cochlear implant 101.

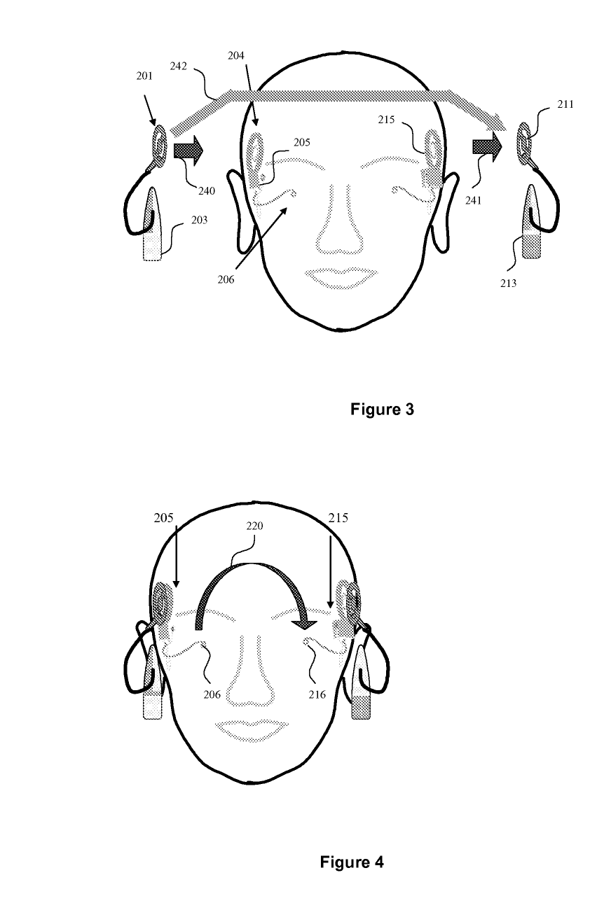

FIG. 3 diagrammatically shows an electromagnetic signal 240 that is used to convey power, data and power from sound processor 203 to its associated implanted component 205. However, electromagnetic signal 204 continues to propagate, and may also induce unwanted artifacts as signal 242 in coils 215, 211 of laterally-opposed cochlear implant 102. The inventor has discovered that the amplitude of these artifacts, when coupled into coil 211 of cochlear implant 102, is sufficient to disrupt the reception of low amplitude data telemetry signals 241 conveyed from implanted coil 215 to coil 211 and hence to sound processor 213. This creates difficulties for efficient and reliable operation of cochlear implant 102, both in respect of the collection of telemetry data and in respect of any operating parameters of the prosthesis which are responsive to the telemetry data.

FIG. 4 is a perspective view of system 100 illustrating another form of interference in bilateral system 100. When stimulus currents are applied to electrode system 206 of implant 205, the current can be conducted through the intervening body tissue and reach electrode system 215 of implanted component 215 of cochlear 102 as a detectable signal. The amplitude of such a signal may be sufficient to confound low amplitude voltage measurements that may be used to determine parameters, for example aspects of the recipient's neural response and characteristics of the electrode-body tissue interface.

The interfering effects may operate in the reverse direction as well, so as to interfere with the contralateral implant, particularly if the prostheses are of the same type and software version.

Cochlear implants typically undertake complex data processing tasks, for example sound data processing, multi-way data communications, power and peripheral systems management, user interfaces, and the internal house keeping of their digital processing engines. The processing within the cochlear implants introduces processing delays between the audio signal and the delivery of the corresponding electrical stimulation. Each prosthesis 101, 102 in bilateral system 100 is subject to differences in processing demands, and in response will have small differences in timing relative to the other prosthesis 101, 102 in system 100 system. Such differences tend to increase over time. As a consequence, the timing differences between the sound signals will not be preserved, and the loss of phase and temporal detail of delivered sound information can adversely effect a recipient's ability to spatially locate the source of incoming sounds.

In accordance with certain embodiments of the present invention cochlear implant 101 and cochlear implant 102 are communicably coupled to each other. In one embodiment, cochlear implants 101 and 102 are networked together as described in US Patent Application No 2003/0036782A1 by Hartley et al, which is hereby incorporated by reference herein. In one embodiment, a continuous connection is provided between cochlear implants 101 and 102.

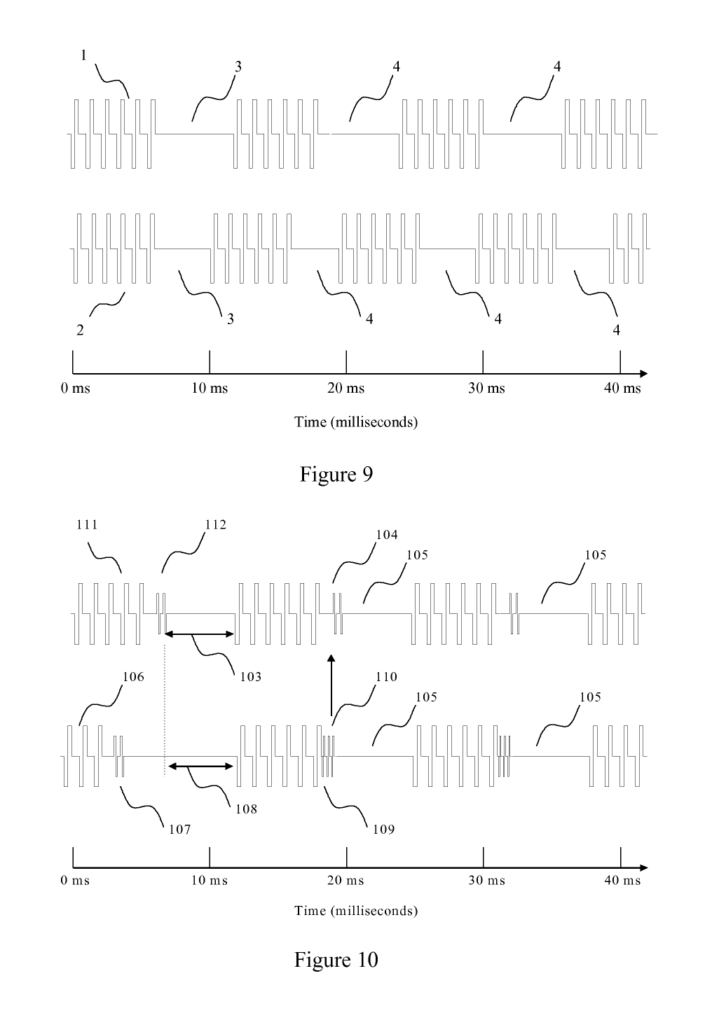

FIGS. 9 and 10 are simplified timing diagrams for hearing prostheses, namely cochlear implants. It should be noted that the repetition period between the stimulus bursts illustrated in FIGS. 9 and 10, which would in practice be approximately 10 ms, and the non-stimulus period of about 4 ms, have been reduced for diagrammatic clarity. In practice, burst repetition periods of around 4 ms are more likely, with these separated by non stimulus periods of a few hundred μs. While the amplitude and periodicity of stimulus pulses is ever changing to reflect the characteristics of conveyed sound information, such changes have been omitted from FIGS. 9 and 10 to further aid clarity.

FIG. 9 is a simplified timing diagram illustrating the timing between two unsynchronized cochlear implants of a bilateral hearing prosthesis system, for one channel of neural stimulation. Graph 1 illustrates bursts of bi-phasic stimulus pulses of varying amplitude from one cochlear implant with a pulse width of around 25 μs per phase and separated by an interval of around 1 ms.

The lower graph 2 contains bursts of biphasic stimulus pulses of varying amplitude from the other cochlear implant in the bilateral hearing prosthesis. These biphasic stimulus pulses have a pulse width of around 25 μs per phase, and are separated by an interval of around 1 ms. Sensitive operations are not subject to potential interference due to emissions from a proximately located hearing prosthesis when those operations are performed when, by random co-incidence only, neither hearing prosthesis 101, 102 is performing interfering operations such as, for example, delivering neural stimulation. Such periods of time are illustrated in the two graphs by reference numeral 3. On the other hand, the inventor has concluded that sensitive operations such as measurements and telemetry may not be reliably performed while interfering operations are being performed by proximate hearing prostheses. Such periods of time are illustrated in the two graphs by reference numeral 4. In this illustrative example, interfering operations comprises the delivery of stimulus currents as such operations deliver stimulus currents which may cause disruptive interference in a proximately located hearing prosthesis. Similarly, the associated high power data communications that invokes the stimulus currents also may cause disruptive interference in a proximately-located hearing prosthesis.

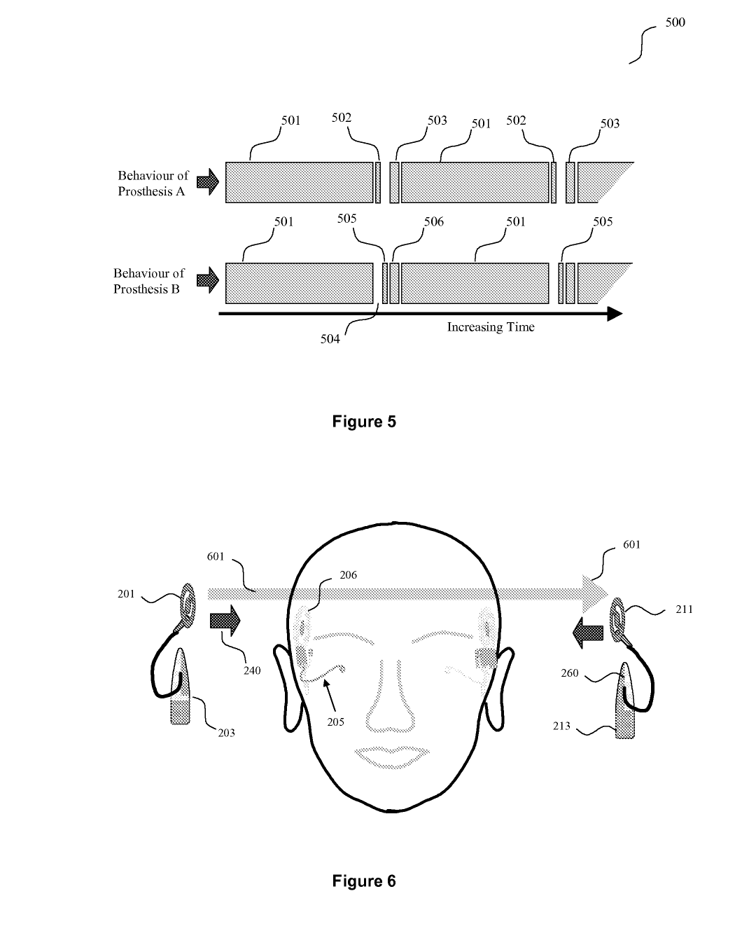

FIG. 5 diagrammatically illustrates in simplified form the temporal behavior of two synchronized, bilaterally disposed cochlear implants A and B. The process by which prostheses A and B become synchronized will be discussed further below. It will be appreciated that a reasonably high degree of synchronization is required in order to achieve the timing relationships which will be described.

As discussed above, the RF signals associated with telemetry being transmitted from, for example, the implanted component to the associated sound processor of cochlear implant A may be negatively affected by the RF signal transmitted by the sound processor to the implanted component of cochlear implant B. This is, in part, because of the much larger transmission power levels used for transmission by the sound processors relative to the return transmission from the implant components.

According to the exemplary implementation shown in FIG. 5, the operation of low power inter-device communications or sensing or measurement of low amplitude signals, such as those associated with the neural response of the recipient, are conducted by cochlear implant A during period 503 and by cochlear implant B during period 506. At this time, neither implant A nor B is delivering stimuli, and stimulus instructions are not being sent to cochlear implant A or B. As such, interference in either direction is unlikely.

It will be understood that the selection of suitable time periods is very specific to device types and operating modes, and would need to be considered for each different hearing prosthesis. However, the basic principle is that when prosthesis A is undertaking interfering operations; that is, those operations which may interference with the sensitive operations being performed by B, then prosthesis B will either pause, or undertake operations which are unlikely to cause the interference (and vice versa).

It should be noted that the timing relationships indicated by FIG. 5 are illustrative of the process rather than an accurate representation of exact timing relationships. The period during which high power data communications and stimulation delivery 501 may be halted to allow synchronizing data to be exchanged and measurements to be undertaken is limited to very small fractions of a second, so as to remain imperceptible to the recipient.

FIG. 10 illustrates, in a view similar to FIG. 9, the timing of two bilaterally implanted cochlear implants synchronized in accordance with the teachings of the present invention, for one channel of neural stimulation. Graph 111 illustrates bursts of biphasic stimulus pulses of varying amplitude from cochlear implant A with a pulse width of around 25 μs per phase and separated by an interval of around 1 ms. Similarly, Graph 106 illustrates bursts of biphasic stimulus pulses of varying amplitude from cochlear implant B with a pulse width of around 25 μs per phase and separated by an interval of around 1 ms. Waveform 112 illustrates two biphasic synchronizing pulses supplied from cochlear implant A with a pulse width of around 10 μs per phase and separated by an interval of around 20 μs and of an amplitude below that which is likely to evoke a hearing percept.

Similarly, waveform 107 illustrates a burst of two biphasic synchronizing pulses supplied from cochlear implant B with a pulse width of around 10 μs per phase and separated by an interval of around 20 μs and of an amplitude below that which is likely to evoke a hearing percept.

Period 103 is the delay period between the delivery of synchronizing pulses and the start of stimulus pulses when no synchronizing pulses from another hearing prosthesis are detected. On detection of waveform 112 by cochlear implant B, cochlear implant B resets its operational sequence timing so as to delay the start of its stimulation delivery and or other functions by a time period that closely approximates period 103. Cochlear implant B acknowledges its synchrony with cochlear implant A by adding a third synchronizing pulse to produce waveform 109, reducing the time interval between them from 20 to 10 μs as well as reducing the time interval between the cessation of stimulus pulses and the start of synchronizing pulses.

On detecting the third synchronizing pulse from cochlear implant B at time 110, cochlear implant A resets its operational sequence control timer such that it almost immediately begins transmitting a pair of synchronizing pulses 104 which in turn and subsequently detected by cochlear implant B. In this mode, both prostheses are synchronized so as to guarantee periods 105 when sensitive measurements and low power telemetry data linking can be undertaken by both devices without interference.

It will be appreciated that many different timing configurations may be employed according to embodiments of the present invention in order to achieve the desired synchronization. The synchronization signals will need to remain detectable. Further, it is preferred that neural stimulation is not interrupted for periods much greater than around 500 μs, as otherwise the interruptions may become perceptible to the recipient.

Synchronization is described above as the approach taken to achieve the timing relationships discussed above. Further, if the stimuli are not presented in suitable synchrony, some of the advantages of bilateral implantation, relating to relative signal timings, phase differences, and signal levels, are lost.

In order to achieve synchronization, some means is required whereby the time dependant operational behavior of the hearing prostheses are synchronized with one another. The short term timing accuracy of the internal clocking oscillators generally employed in cochlear implants may allow the prostheses to run in synchrony for periods of a few seconds, but not for the many hours required for normal use of a hearing prosthesis. To ensure synchrony is maintained for a long period of time, some mechanism for establishing and maintaining synchronization is implemented.

In one embodiment, the prostheses are physically connected by a cable or the like, and use a common clocking signal, or similar continuous timing control. Such an arrangement is disclosed in US patent application No 2003/0036782A1 by Hartley et al, the disclosure of which is hereby incorporated by reference.

Alternatively or additionally, certain embodiments of the present invention implement using a synchronization method which provides a periodic signal to allow synchronization to be attained. Such an arrangement is more practical, for example where a hearing prosthesis is fully implanted, and in principle requires less power to implement. It also allows each prosthesis to operate independently without any action by the recipient, for example in case of a fault in one of the prostheses.

According to one such implementation, hearing prosthesis A repetitively transmits a signal that is detectable by hearing prosthesis B in a manner that allows hearing prosthesis B to synchronize its operational behavior with that of prosthesis A, and vice versa.

Considering FIG. 5, prosthesis B detects valid synchronizing signals 502 from prosthesis A. Prosthesis B then repetitively imposes a time delay 504 prior to the transmission of its own synchronizing signal 505, as well as modifying characteristics of this signal so as to convey confirmation as to its state of synchrony, to prosthesis A. Once the two prostheses are synchronized as shown in FIG. 5, stimuli can be applied by both prostheses at the same time during period 501.

While the following described embodiments of the invention operate on the basis of a need to maintain synchrony in a more or less continuous manner (despite only periodically sending synchronization signals), configurations of the invention are never the less able to function in an intermittent manner so as to provide synchrony only for periods when it is particularly beneficial to a recipient. Such an operating mode may, for example, reduce the electrical power consumed so as to conserve battery power.

In one embodiment of the invention as illustrated in FIG. 6, a synchronizing signal 601 generated by prosthesis A is embedded into, or otherwise added to the stream of wireless power and data transmissions 240 that are transmitted by coil 201 to implanted component 205. This signal is also received by coil 211, or some other induction coil 260 such as a telecoil, for detection and processing within the B sound processor 213.

Once a sequence of two or more signals matching the acceptance criteria of valid synchronizing signals are detected, internal clocking and event sequence control circuits of the receiving member are reset or synchronized repetitively with each valid synchronizing signal that follows. An example of this has been described in more detail with respect to FIG. 10.

As will be apparent to those of ordinary skill in the art, these synchronizing signals and the anticipated timing of their detection may be timed to occur at a fixed rate using such circuitry as phase locked loops, or made to occur at varying rates as might be controlled using pseudo randomly generated timing sequences.

According to this implementation, once the synchrony of one prosthesis is maintained consistently for the required synchronizing period the synchronizing signal transmitted by that prosthesis is modified so as to alert the other prosthesis as to its state of synchrony. The actual number of detection events could be just one or two, or several depending on the signal and noise levels observed in practice and their effect on synchronizing signal detection reliability. This parameter could be fixed, programmable or self adjusting to suit conditions in vivo. In this way, the first member of an identical pair of prostheses to become synchronized is slaved to the operation of the other, which in this case can be thought of as the master timing control member. Although synchronized, time delay offsets ensure that the operational behavior of each member is timed so as not to clash with that of their bilateral counterpart. This master-slave timing control relationship continues as described until either prosthesis is turned off, removed or disrupted in some way. Synchrony is automatically restored in the manner described when operation of both prostheses is returned.

It will be understood that numerous variations to the above regime may be used to achieve and maintain synchrony in a manner that is more energy efficient or less vulnerable to external interference. For example, an alternating technique whereby the master-slave timing relationship referred to above is repetitively alternated in some manner might be employed. The use of pseudo randomly timed synchronizing transmissions could also reduce the time required to achieve synchrony between the prostheses.

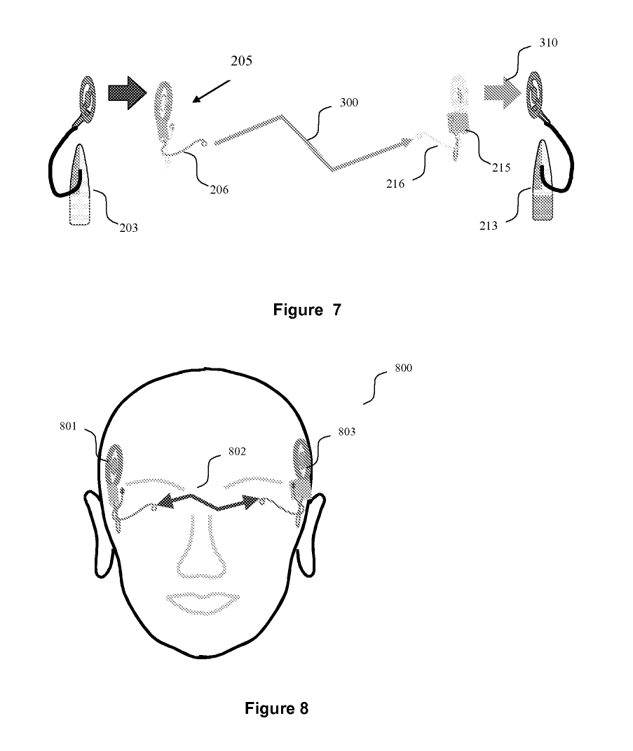

A second embodiment of the synchronization arrangement is illustrated in FIG. 7. In this case, the external part of one prosthesis 203 of a bilaterally disposed system repetitively conveys data to its implanted part 205. This causes the implanted part to subsequently convey repetitive signal currents 300 via its electrode system 206. In a suitable mode, for example when the other prosthesis is configured to detect neural response, this signal can be detected by the electrode array 216 of the other prosthesis, and hence conveyed to the speech processor 213. The currents conveyed have specific timing and amplitude characteristics to ensure that they remain biologically safe, and at a level insufficient to evoke any sensation of hearing for the recipient. These signals may also have characteristics that make them easily distinguishable from any applied neural stimulus and resulting neural response signals. The timing controls previously discussed provide a window for such signal to be sent and received. It is preferred that the timing information is preserved through the use of fast response synchronization signal detection circuitry with constant response time.

Once the repetitive signal voltages are received at the electrode system 216 of implant 215, data signals 310 describing this detection and timing are conveyed using wireless telemetry to the corresponding sound processor 213. Synchrony of this prosthesis 231 with the other device 203 is now affected in much the same manner as has been previously described.

As in the first embodiment, either prosthesis may become master or slave depending on which prosthesis falls into synchrony first.

As will be apparent to those skilled in the art, other embodiments of the invention are possible by applying a wide variety of timing and amplitude techniques to attain a desired or required degree of synchrony to enable the hearing prostheses to control the timing of the sensitive and interfering operations.

Another exemplary synchronizing regime may be applied on an “only as needed basis”, whereby prostheses configured to operate independently for much of the time, transmit signals to alert the other of the need to operate synchronously for some predetermined time period or until other transmissions signal a return to independent operation. This part time use of synchrony allows the extra battery power required for synchronous operation to be conserved for use only when needed or most useful.

The specific timing signal examples described previously can, for example, be embedded, form part of, or be derived directly from the stimulus and data signals used during normal operation of the implementing hearing prostheses. Synchronizing signals as well as the master slave relationship referred to previously may be alternated intermittently, randomly, or continuously at various rates.

In another embodiment, the synchronizing information is exchanged between the prostheses by way of largely continuous, but modulated oscillatory electromagnetic signals. The modulation might be performed in any suitable manner, for example amplitude, phase, frequency, and or frequency shift keying, or combinations thereof.

In this way, two bilaterally disposed, partly or totally implanted prostheses 801 and 803 could, as is shown in FIG. 8, share incoming sound information from each others microphone (802) in a manner that allows beam forming and other signal processing techniques to be used to provide enhanced signal processing. This feature can benefit a recipient's ability to discriminate sounds from a particular source in a manner that improves speech perception in noisy environments.

Further, this arrangement may be used to allow each sound processor select either microphone (the A or B side), or a combination thereof, as the basis for processing. Other information or data may also be conveyed or shared between prostheses. The manual adjustment of a control setting of one external component may be conveyed so as to replicate the same setting of an opposing.

It will be appreciated that the above exemplary embodiments have en described in the context of a two prosthesis system; additional hearing prostheses may be similarly co-ordinate. For example, each prosthesis may have several components which need to work together on a common timing basis, for example for effective communications, and this coordination may be performed within the components of each prosthesis. The matters which are sensitive or interfering may differ between devices, based upon the way they are connected and interact. The general principles of the present invention may be applied to partly or fully implanted systems, with different splits in functionality relative to conventional hearing prostheses as described.

In addition to the use of electric or electromagnetic signals described previously, other means can be used to achieve the said synchrony. The present invention is not limited to any specific mechanism for achieving synchrony.

A more or less continuous detection of certain types of abrupt sound elements by each bilateral member could be used to achieve some limited degree of synchrony. Certain vocal sounds of a recipient, on reaching the similarly located microphone of each bilateral member would be delayed by more or less the same time such that these sounds could be used to synchronies their operation.

Hearing prostheses that employ mechanical vibratory means to evoke or enhance a recipient's hearing may be synchronized through the sharing of synchronizing data conveyed as sound through the air, or as vibration conveyed through a recipient's body. This data could be conveyed at very low or very high acoustic frequencies such that it would remain inaudible to the recipient and other persons.

It will be appreciated that the present invention may be applied with numerous variations to the embodiments described, and with the addition of further features.

Further features and advantages of the present invention may be found in Australian Provisional Application No. 2007902247, entitled “Bilateral Prosthesis Synchronisation,” and filed Apr. 30, 2007, which is hereby incorporated by reference herein.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A method for operating a bilateral hearing prosthesis having first and second hearing prostheses, the first hearing prosthesis configured to perform at least one operation sensitive to interference by emissions generated by the second hearing prosthesis when the second hearing prosthesis performs at least one interfering operation, the method comprising:
synchronizing timing of the sensitive and interfering operations performed by the first and second prostheses; respectively; and
performing, based on said synchronized timing, the sensitive and interfering operations such that relative timing of said performance of the sensitive and interfering operations minimizes the interference of the sensitive operations by the emissions generated by the second hearing prosthesis.

2. The method of claim 1, wherein the sensitive operations comprise:
detecting neural response.

3. The method of claim 1, wherein the sensitive operations comprise:
measuring electrode impedances.

4. The method of claim 1, wherein the sensitive operations comprise:
transmitting telemetry data.

5. The method of claim 2, wherein the interfering operations comprise:
transmitting power to an implanted component of the hearing prosthesis.

6. The method of claim 2, wherein the interfering operations comprise:
transmitting data to an implanted component of the hearing prosthesis.

7. The method of claim 1, wherein synchronizing timing comprises:
transmitting timing information between the first and second prostheses using continuous signals.

8. The method of claim 1, wherein synchronizing timing comprises:
transmitting timing information between the first and second prostheses using periodic transmissions of synchronizing signals.

9. The method of claim 8, wherein synchronizing timing comprises:
transmitting between the first and second hearing prostheses a signal indicating successful synchronization of the first and second hearing prostheses.

10. The method of claim 1, wherein at least one of the first and second hearing prosthesis comprises a cochlear implant.

11. A hearing prosthesis including a receiver for receiving signals, said receiver communicating with a processor, said processor being operatively adapted to detect synchronizing signals within the received signal, and in response to the synchronizing signals, adjust the timing of operations performed by the prosthesis so as to establish a co-ordinate timing regime with a bilaterally disposed prosthesis.

12. A hearing prosthesis according to claim 11, wherein the co-ordinate timing regime is such that sensitive operations and interfering operations are undertaken by both the prosthesis and the bilateral prosthesis at substantially distinct times.

13. A method of communicating between active implanted devices, each said device including a stimulation device adapted to deliver electrical stimuli to the body, and to sense electrical responses to stimuli, wherein the stimulation device is further adapted to send signals which carry information for detection by another implanted device.

14. A method according to claim 13, wherein the active devices are cochlear implants, the stimulation device is an intracochlear array, and the signals are stimuli delivered by the intracochlear array at levels below those likely to be perceived as sound by a recipient.

15. A method of communicating between two active implanted devices, wherein such communication is affected by the transmission and reception of vibratory or acoustic signals conveyed through the air and/or body tissues of the recipient.
